(19) United States
(12) Patent Application Publication
(10) Pub. No.: US 2026/0369170 A1
(43) Pub. Date: Nov. 19, 2026

(54) METHOD FOR MEASURING THE CONTENTS OF AT LEAST ONE GAS EMITTED BY A SOURCE IN A PLUME PROPAGATING THROUGH THE ATMOSPHERE FROM THE SOURCE, AND ASSOCIATED METHOD AND DRONE

(71) Applicant: TotalEnergies OneTech, COURBEVOIE (FR)

(72) Inventors: Nicolas GALAS, SOLAIZE (FR); Ludovic DONNAT, SOLAIZE (FR)

(73) Assignee: TotalEnergies OneTech, COURBEVOIE (FR)

(21) Appl. No.: 19/133,700

(22) PCT Filed: Nov. 29, 2023
(86) PCT No.: PCT/EP2023/083605

§ 371 Date: May 28, 2025

(30) Foreign Application Priority Data
Nov. 30, 2022 (FR) FR2212559

(51) Int. Cl. G08G 5/34; B64U 101/35; G01N 33/00; G08G 5/32

(52) U.S. Cl. CPC G08G 5/34; G01N 33/0073; G08G 5/32; B64U 2101/35

(57) ABSTRACT

A method for measuring contents of a gas emitted in a plume includes: defining an initial flight plan of a drone following an initial measurement area comprising a plurality of initial measurement segments; flying the drone according to the initial flight plan and measuring data representative of content on at least one initial measurement segment During implementation of the initial flight plan and/or after the implementation of the initial flight plan the method may further include generating a modified flight plan from the data representative of content measured over the at least one initial measurement segment. The modified flight plan comprises a plurality of modified measurement segments defining a modified measurement area, and the method includes measuring the data representative of content over the plurality of modified measurement segments of the modified measurement area.

### CROSS REFERENCE TO RELATED APPLICATIONS

The present application is a U.S. National Phase Application under 35 U.S.C. § 371 of International Patent Application No. PCT/EP2023/083605 filed Nov. 29, 2023, which claims priority of French Patent Application No. 2212559 filed Nov. 30, 2022. The entire contents of which are hereby incorporated by reference.

### FIELD

This invention relates to a method for measuring the contents of at least one gas emitted by a source in a plume propagating through the atmosphere from the source. The method includes the collection of data representative of the content of at least one gas in the atmosphere away from the source by flying a drone equipped with a measuring unit for data representative.

### BACKGROUND

The gases to be measured are in particular greenhouse gases such as methane or carbon dioxide.

Environmental protection concerns have contributed to the strengthening of legislation on pollutant emissions, particularly in Europe.

### SUMMARY

As a result, industrial units, such as those in the oil or chemical industry, must adapt to increasingly stringent environmental constraints.

In particular, greenhouse gases are emitted during operating activities, transport, refining, and storage of hydrocarbons. These emissions are monitored by operators and are regularly subject to reduction measures.

It is particularly necessary to characterize which sources of greenhouse gases exist and what quantities are emitted by these sources to ensure the control thereof and to report progress made.

However, the identification of greenhouse gas emission sources and the quantification of diffuse and fugitive emissions do not provide complete satisfaction.

Indeed, emissions are very difficult to measure because they are often unchanneled and potentially near basins or lakes or in inaccessible places, e.g. at height or in the middle of a unit.

A major difficulty in evaluating emissions from a point source within a facility is often the difficulty, or even the impossibility, of getting close enough to the source to measure the gas flow emitted by the source into the atmosphere. Moreover, given the wind, the gas flow generated by the source disperses and propagates in the atmosphere in the form of a plume. It is therefore generally difficult and imprecise to measure emissions emitted by a point source when moving away from the source.

### DETAILED DESCRIPTION

To address this problem, it is known, e.g., from US 4,135,092 to determine an approximate wind direction and to circulate a gas content measurement system following a rising scale by describing successive segments over a width and height assumed to exceed the dimensions of the plume.

Such a method gives satisfaction, especially when the wind direction is well determined, and the progression of the plume is sufficiently homogeneous to evaluate in advance what are the width and height thereof.

However, the accuracy of the emitted flow measurement depends on the ability of the measurement system to sweep an entire section of the plume through a given measurement plane to integrate all the concentrations present in the plume.

If the measurement plane is not well positioned, or if the plume extends beyond the predefined rising scale, the measurement becomes imprecise. Indeed, a portion of the gas contents emitted by the source present in the plume is not measured, and the calculated flow is lower than the emitted flow.

An aim of the invention is to obtain a method for measuring emissions from a source producing a plume, with increased accuracy, even if the wind direction or the extent of the plume is poorly evaluated initially.

To this end, the invention relates to a method of the aforementioned type, characterized by the following steps:
definition of an initial flight plan for the drone following an initial measurement area comprising a plurality of initial measurement segments;
flight of the drone following the initial flight plan and measurement of data representative of content over at least one initial measurement segment,
during the implementation of the initial flight plan and/or after the implementation of the initial flight plan, generation of a modified flight plan from the data representative of content measured during at least one initial measurement segment, the modified flight plan including a plurality of modified measurement segments defining a modified measurement area,
measurement of data representative of content over the plurality of modified measurement segments of the modified measurement area.

The method according to the invention may comprise one or more of the following characteristics, taken alone or in any technically possible combination:
the modified flight plan is generated during the implementation of the initial flight plan, with the drone flying along at least one modified measurement segment replacing an initial measurement segment or adding to an initial measurement segment;
the modified measurement segment replacing an initial measurement segment extends the initial measurement segment beyond an end of the initial measurement segment until the value of at least one datum representative of content measured in the extension of the initial measurement segment is below a background level threshold value;
the method comprises the generation of a new measurement segment extending adjacent to the ongoing measurement segment as long as at least one datum representative of content over the newly generated measurement segment is above a background level threshold value;
the new measurement segment is located above or below the ongoing measurement segment;
the gap between the ongoing measurement segment and the new measurement segment is adjusted based on the estimated distance from the source, and/or based on the extent of the measurement segments previously flown by the drone;
the generation of the modified flight plan is implemented after the complete execution of the initial flight plan, with the drone remaining in flight between the end of the execution of the initial flight plan and the execution of the modified flight plan;
when the data representative of content measured over the initial measurement area have values within an exploitable value range, the generation of the modified flight plan comprises determining the barycenter of the values of data representative of content measured over the initial measurement area, and advantageously measuring wind data at each measurement point of a datum representative of content, then defining the measurement area from the position of the barycenter and advantageously a mean wind direction calculated from the wind data determined at each measurement point;
when the data representative of content measured over the initial measurement area have at least one value above a saturation threshold value, the generation of the modified flight plan includes positioning the modified measurement area further from the source than the initial measurement area;
when the data representative of content measured over the initial measurement area all have a value below a background level threshold value, the generation of a modified flight plan includes estimating a wind direction measured from wind data measured during the flight following the initial measurement area, and determining an angle between a measured wind direction and an initially assumed wind direction during the definition of the initial measurement area;
when the angle between the measured wind direction and the assumed wind direction is greater than a given coherence angle, the generation of the modified flight plan includes modifying an orientation of the modified measurement area with respect to the source compared to the orientation of the initial measurement area with respect to the source or when the angle between the measured wind direction and the assumed wind direction is less than a given coherence angle, the generation of the modified flight plan includes defining a modified measurement area closer to the source than the initial measurement area;
the measurement segments are lines inscribed in straight lines, the measurement area being a measurement plane;
the position of the initial flight area is determined from measured wind data and a dispersion calculation performed from the measured wind data;
the measured wind data is acquired from a wind measurement sensor carried by the drone, during a warmup phase of the drone and/or during an ascent of the drone to a starting altitude for the implementation of a measurement segment of the initial flight plan;
the steps of defining the initial flight plan and generating the modified flight plan are implemented by a control unit for the movement of the drone on-board the drone or at least partially remote from the drone;
the measurements of data representative of content of at least one gas present in the atmosphere are carried out by a measurement sensor of representative data specific to measuring data representative of content of at least two gases present in the atmosphere, the measurement sensor of representative data including at least one measurement cell open to the atmosphere, and for the or each measurement cell, at least one first laser source specific to injecting into the measurement cell, a first laser beam at a first wavelength characteristic of a first gas to be detected and a second laser source specific to injecting into the measurement cell a second laser beam at a second wavelength characteristic of a second gas to be detected, the measurement sensor of data representative including at least one detector, the detector(s) being specific to capturing a first measurement signal from the measurement cell resulting from the injection of the first laser beam into the measurement cell and a second measurement signal from the measurement cell resulting from the injection of the second laser beam into the measurement cell;
the measurement cell includes two mirrors located opposite each other and spaced apart, defining a measurement cavity between them, and two supports carrying the two mirrors respectively, the laser sources and the detector being assembled on the supports, spaced from the measurement cavity.

The invention also relates to a method for measuring emissions from a source, comprising the implementation of a measurement method as defined above, then the calculation, by a flow calculation system, of a gas flow emitted by the source from the data representative of content measured over the modified measurement area during the execution of the modified flight plan.

The invention also relates to a drone for measuring the contents of at least one gas emitted by a source in a plume propagating through the atmosphere from the source,
the drone including a measurement unit of data representative of content of at least one gas in the atmosphere away from the source, characterized by a control unit for the movement of the drone movement configured:
to define an initial flight plan for the drone following an initial measurement area comprising a plurality of initial measurement segments;
to control the flight of the drone following the initial flight plan to measure data representative of content over at least one initial measurement segment,
during the implementation of the initial flight plan and/or after the implementation of the initial flight plan, to generate from a modified flight plan from the data representative of content measured during at least one initial measurement segment, the modified flight plan including a plurality of modified measurement segments defining a modified measurement area,
to control the flight of the drone to measure data representative of content over the plurality of modified measurement segments of the modified measurement area.

### BRIEF DESCRIPTION OF THE DRAWINGS

The invention will be better understood, based on the following description, given solely by way of example, and made in reference to the appended drawings, in which:

FIG. 1 is a schematic view of a first measurement kit comprising a drone according to the invention, intended for the implementation of the measurement method according to the invention;

FIG. 2 is a view of a gas source within a facility, and the plume emitted by the gas source;

FIG. 3 is a view of the adaptive flight plan implemented by the drone of the kit of FIG. 1 following the method according to the invention;

FIG. 4 is a flowchart illustrating the initial steps of determining an initial flight plan, following an initial flight plan describing an initial measurement area;

FIG. 5 illustrates the implementation of a second method according to the invention, for correcting the initial flight plan, in the case of gas content detection without saturation, in the case of gas content detection with saturation, and in the case of absence of gas content detection;

FIG. 6 illustrates a step of determining a corrected measurement area in the case of gas content detection without saturation;

FIG. 7 is a view similar to FIG. 6, in the case of gas content detection with saturation;

FIG. 8 is a view similar to FIG. 6, in the case of absence of gas content detection, with a wind direction inconsistent with that initially determined;

FIG. 9 is a view similar to FIG. 6, with a wind direction consistent with the position of the initial measurement area; and

FIG. 10 is a schematic view of a control unit of the drone according to the invention.

### DETAILED DESCRIPTION

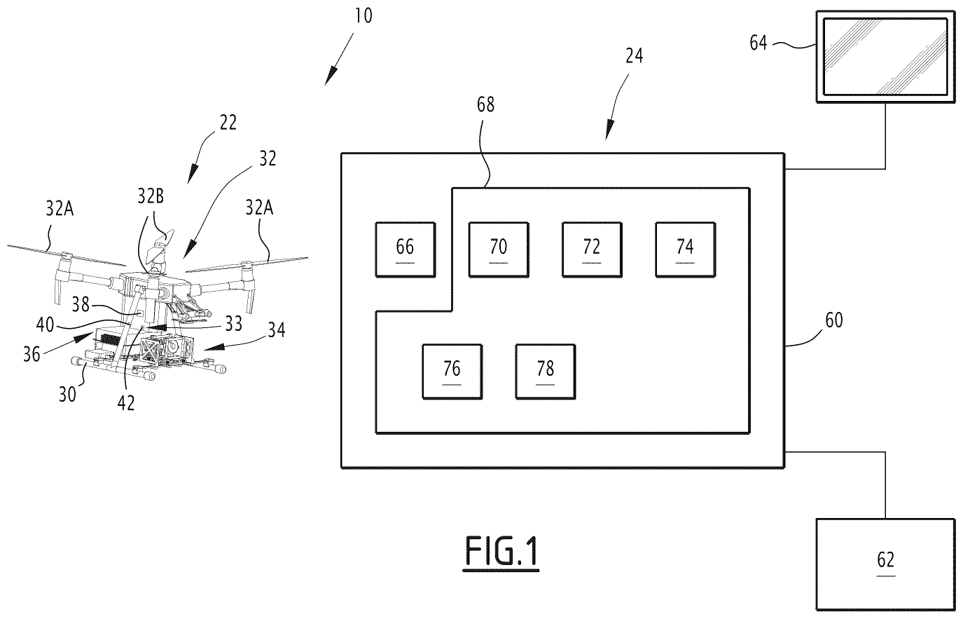

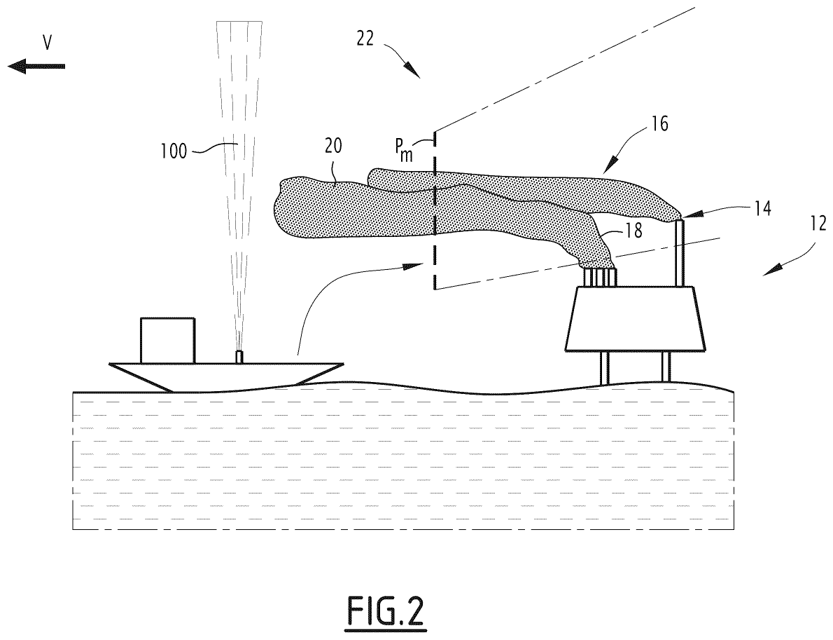

A measurement kit 10 for emissions of at least one gas emitted by a source into the atmosphere is schematically illustrated in FIG. 1. The kit 10 is intended to implement a method for measuring emissions from an industrial facility 12, schematically represented in FIG. 2.

Preferably, the emission of at least two gases present in the atmosphere is measured by the method according to the invention. The gases are preferably methane and carbon dioxide.

In variants, other gases are measurable, such as aromatic gases, in particular benzene or even 1,3 Butadiene, carbon monoxide, ethane, and more generally Volatile Organic Compounds.

The industrial facility 12 is in particular an oil facility, particularly an operating, transport, refining, treatment, or storage facility for hydrocarbons located at sea or on land. The facility 12 includes at least one source 14 emitting gases the content of which is measured.

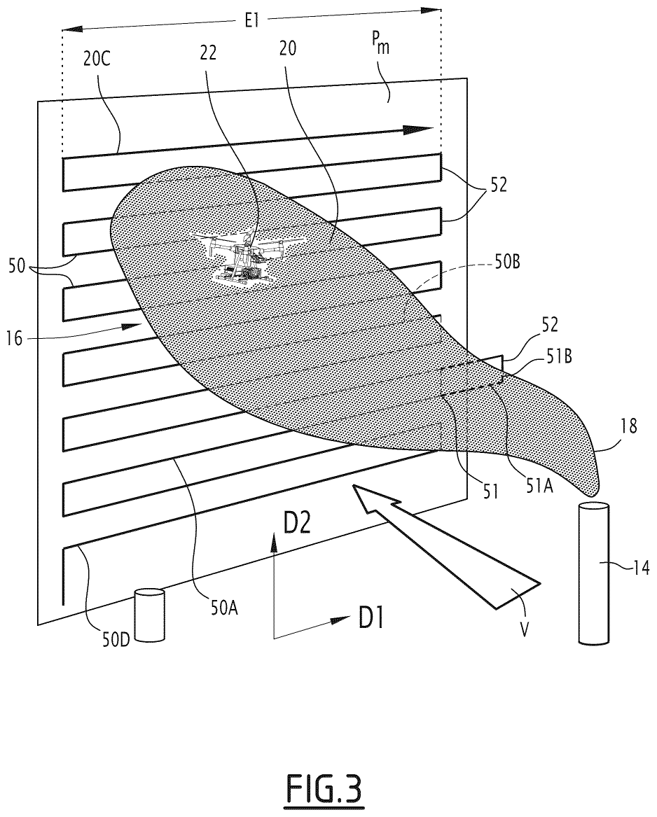

In the example shown in FIGS. 2 and 3, the source 14 is a flare. Said flare emits gases following a plume 16 that emanates from the source 14 and propagates under the effect of the wind V.

The plume 16 is driven by the wind V circulating in the atmosphere neighboring the source 14. It advantageously has a zone 18 of elevation of the plume 16 that is substantially vertical and a zone 20 of propagation of the plume, which in this example is substantially horizontal.

Referring to FIG. 1, to implement the measurement method, the measurement kit 10 includes a drone 22 for collecting data representative of content of at least one gas, preferably at least two gases, at a plurality of positions in the atmosphere, away from the source 14.

The kit 10 further includes a calculation system 24, capable of implementing a method for calculating a flow of each gas emitted by the source 14 in the atmosphere, from the data representative content of each gas into the atmosphere measured by the drone 22.

The drone 22 is capable of performing the necessary measurements to collect data representative of content of at least one gas present in the plume 16, away from the source 14. It includes a chassis 30, a propulsion set 32, capable of allowing the chassis 30 to take off above and away from the ground and move in flight in the atmosphere above the ground.

The drone 22 further includes a measurement unit 34, a control set 36 for the measurement unit 34, and preferably, a remote transmission system 38.

Referring to FIG. 1, the propulsion set 32 includes a plurality of propulsion organs 32A, which here are propellers driven in rotation by a motor.

The propulsion set 32 further comprises an energy source 32B formed here by a battery and a system 33 for locating and controlling the movement of the drone 22 in the atmosphere.

In this example, the drone 22 is a rotary wing multi-rotor drone. It is wingless, the lift thereof being provided by the propulsion set 32.

The drone 22 is, e.g., a rotary wing quadcopter drone, in particular a DJI M200 drone marketed by DJI.

Alternatively, the drone 22 is a drone (e.g. marketed under the name Tundra by Hexadrone), equipped with an automatic navigation system, capable of flying autonomously. Such a drone is then capable of autonomously managing flight safety, particularly concerning the authorized flight domain, and traffic and obstacle management.

The propulsion set 32 includes a plurality of propellers rotating around substantially vertical axes. By “substantially vertical,” it is generally meant that the axes of rotation of the propellers are inclined by less than 30° relative to the vertical.

When the propeller motors are electrically powered by the battery, the propellers are driven in rotation around their axis, generating a downward airflow.

The localization and control system 33 includes a position sensor 40, in particular a GPS and/or an inertial unit. The control system further comprises a control unit 42, capable of piloting the movement of the drone 22 along a trajectory defining a measurement area comprising parallel measurement segments connected by connection segments.

Preferably, for the implementation of the measurement method, the drone 22 is capable of performing each flight plan following a rising scale movement, as illustrated by FIG. 3.

In this case, the measurement area is contained in a plane, and the measurement segments and connection segments are straight lines 50.

The drone 22 then moves along a plurality of lines 50 parallel to a first direction D1, with a connection segment 52 between each pair of adjacent parallel lines 50. The connection segment 52 is performed following a second direction D2 transverse to the first direction D1.

Here, the first direction D1 is a horizontal direction, and the second direction D2 is a vertical direction.

In this example, all the parallel lines 50 swept by the drone 22 extend substantially in the same vertical measurement plane Pm.

The extent E1 of the lines 50 following the first direction D1 is chosen in an initial flight plan based on the width of the plume 16, to sweep the entire plume 16. This extent E1 is generally greater than 20 m and is between 20 m and 500 m.

The distance between the lines 50 is defined by an extent E2 of the connection segments 52 following the second direction. This extent E2 is, e.g., greater than 1 m and in particular between 1 m and 50 m.

In a first embodiment, in manual piloting, the control unit 42 is capable of receiving movement instructions remotely and manually via a remote control.

In another embodiment, which may complement the first, the control unit 42 is autonomous. It is capable of receiving at least one initial flight plan following an initial measurement area, to pilot the movement of the drone 22 following this initial flight plan, and according to the invention, to automatically adapt/correct the initial flight plan during the implementation of the initial flight plan, or after its implementation.

The measurement unit 34 includes at least one sensor capable of performing measurements of data representative of content of at least one gas present in the atmosphere, at a plurality of points along each line 50.

Preferably, the data representative of content of at least two gases is collected by the measurement unit 34 along each line 50.

The measurements are performed continuously along the line 50. The measurement frequency of data representative of content data in each gas content is, e.g., greater than 1 Hz and is in particular between 1 Hz and 100 Hz.

The wind direction is preferably measured directly on the drone 22 at each measurement point, e.g. by an anemometer.

An example of a measurement unit 34 is described in the application WO2021191360.

The control system 33 includes a datum collection unit that comprises at least one memory capable of storing the data representative of content of each gas, associated with the geographical position along each line 50, and the wind direction when measured.

The data collection unit is connected to the remote transmission system 38 to allow the export of data to the calculation system 24, during the flight of the drone or after the flight of the drone.

According to the invention, the control unit 42 is positioned in the drone 22, or is at least partially remote from the drone 22.

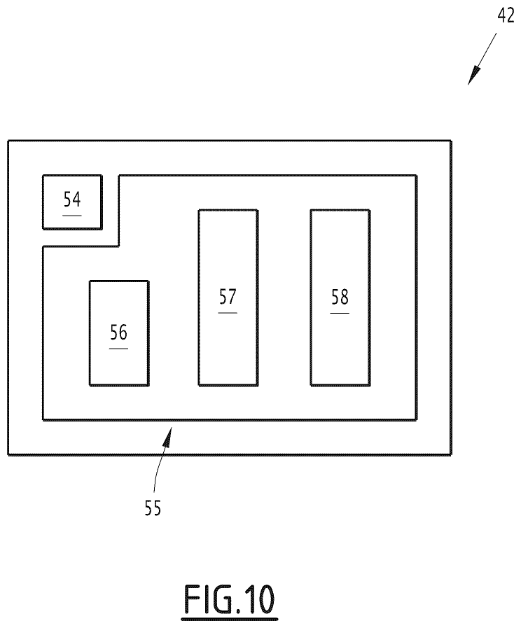

Referring to FIG. 10, the control system generally includes at least one computer including at least one processor 54, and at least one memory 55 containing software modules capable of being executed by the processor 54. Alternatively, the computer includes programmable logic components or dedicated integrated circuits, intended to perform the functions of the modules described below.

In particular, according to the invention, the memory 55 of the control unit 42 includes a module 56 for acquiring and/or calculating an initial flight plan describing an initial measurement area, a trajectory correction module 57 during the execution of the initial flight plan, and/or a trajectory correction module 58 after the execution of the initial flight plan, the modules 57, 58 being intended to generate a modified flight plan following a modified flight area from measurements made of data representative of the content during the implementation of the initial flight plan.

In one embodiment, as described below, the acquisition module 56 is advantageously configured to acquire positioning data of the source 14, in particular geographical coordinates of the source 14, and wind direction data, in particular the angle of the wind direction relative to the source 14. The acquisition module is also capable of acquiring and/or calculating data on the assumed maximum height and width of the plume 16.

The acquisition module 56 is also configured to define the initial trajectory following the initial flight plan, including the width of the measurement segments, the height thereof and the spacing thereof.

During the execution of the initial flight plan, the trajectory correction module 57 is intended to allow for the capture of an entire section of the plume 16.

To this end, it is configured, during the implementation of each measurement segment of the initial trajectory, to collect the data representative of content measured by the measurement unit 34, and after each measurement, to compare the obtained value for the data representative of content to a background level threshold value, corresponding to a non-significant detection of the gas content being measured.

The background level is defined for each gas whose content is being measured. It corresponds, e.g., for methane to a concentration of about 2 ppm.

Alternatively, as described in patent application FR3109217, the background level is determined for each line 50, by calculating a statistical value, e.g., the average of contents measured on line 50, then eliminating the contents above the average value, and repeating the previous steps until the difference between two successive average values is below a convergence threshold.

At each measurement of data representative of content, as long as the value of the data representative of content is above the background level threshold value, the trajectory correction module 57 moves the drone 22 on the measurement segment to perform a new measurement of data representative of content over the same measurement segment at a position adjacent to the position just measured.

This is particularly the case if the representative data at an end 51 of the initial measurement segment 50A provided in the initial flight plan have a value above the background level threshold value.

Referring to FIG. 3, the trajectory correction module 57 defines a modified trajectory that extends the initial measurement segment 51 over a section 51A beyond the end 51 of the initial measurement segment 50A, as long as the value of the data representative of content is above the background level threshold value.

Conversely, if at least one representative datum of the content measured is below the background level threshold value, preferably, if a plurality of successive data representative contents are below the background level threshold value, then the trajectory correction module 57 is configured to stop the movement of the drone 22 over the section 51A and stop the measurement by the measurement unit 34 over this measurement segment 50A.

This thereby defines a new lateral end 51B of the measurement segment 50A modified by the addition of the section 51A.

The trajectory correction module 57 is then configured to move the drone 22 vertically following a connection segment 52 to reach a different altitude and traverse a new measurement segment 50B adjacent to the previous measurement segment 50A.

The spacing between the new measurement segment 50B and the previous measurement segment 50A is determined based on the assumed size of the plume 16 and the distance to the source 14. This spacing is advantageously always greater than one meter, in particular two meters.

The trajectory correction module 57 is configured to repeat the addition of new measurement segments 50B, even beyond the initially planned height of the highest measurement segment planned in the initial flight plan, as long as at least one value of data representative of content over the measurement segment 50B is above the background level threshold value. Thereby, the initial flight plan is modified by the trajectory correction module 57 to add new measurement segments above (or below) segments planned in the initial flight plan to capture the entire section of the plume 16 in the measurement plane Pm.

On the other hand, if all the values of the data representative of content are below the background level threshold value over a given measurement segment, the trajectory correction module 57 is configured to stop the movement of the drone 22 and no longer perform measurements on new measurement segments 50B.

Thus, the trajectory correction module 57 is configured to define, during the execution of the initial flight plan, a modified trajectory that fully bounds the section of the plume 16, even if it was not completely covered in the initial flight plan.

In each measurement segment of the modified flight plan, the ends 51B of each measurement segment 50A, 50B have a value of the data representative of content that is below the background level threshold value, thus covering the entire width of the section of the plume 16.

The upper segment 50C and advantageously the lower segment 50D of the modified flight plan also have values of data representative of content that are below the background level threshold value, thus covering the entire height of the section of the plume 16.

The trajectory correction module 58 after the execution of the initial flight plan is capable of operating in different modes of operation.

In a first mode of operation, in the case where the implementation of the initial flight plan following the initial measurement area has allowed obtaining values of data representative of content above the background level threshold value, and no data representative of content above a saturation threshold value, then the trajectory correction module 58 is configured to calculate the position of a barycenter of the obtained values of data representative of content.

When the drone 22 is equipped with a wind sensor, the trajectory correction module 58 is also configured to determine the wind direction at each measurement point, and possibly to determine the measurement segments that are incomplete if the trajectory correction module 57 was not activated during the implementation of the initial flight plan.

A measurement segment is incomplete when the value of data representative of content at the end of the measurement segment is above the background level threshold value.

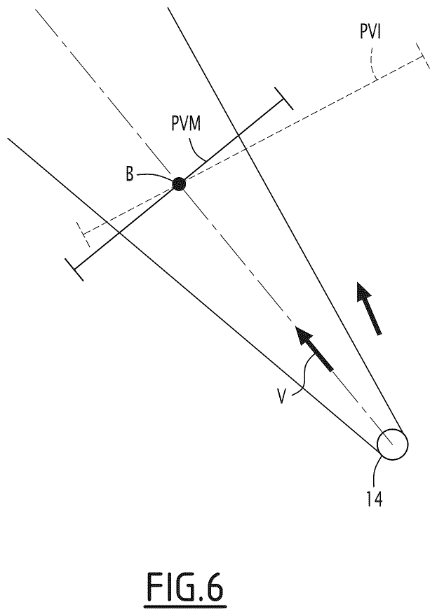

Based on the calculated position of the barycenter, advantageously the wind direction data and the position of the incomplete segments, the trajectory correction module 58 is configured to define a modified flight plan wherein the assumed center position of the section of the plume 16 (by placing the center position, e.g., at the determined barycenter position and preferably keeping the same distance to the source 54), the height, the width, the angular orientation of the measurement area and/or the vertical spacing of the measurement segments are modified, as will be described below with reference to FIG. 6.

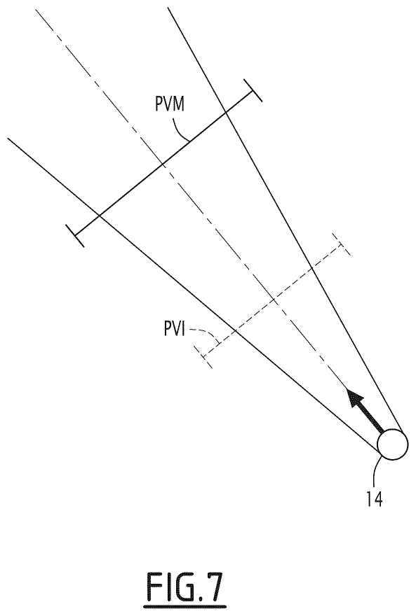

In a second mode of operation, if one or more data representative of content measured during the execution of the initial flight plan are above the saturation threshold value, the trajectory correction module 58 is configured to redefine a modified flight plan, over a modified measurement area further from the source 14 than the initial measurement area, as will be described below with reference to FIG. 7.

In a third mode of operation, when the values of the data representative of content are all below the background level threshold value, the trajectory correction module 58 is capable of determining a wind direction measured throughout the implementation of the initial flight plan.

The trajectory correction module 58 is then configured to calculate a mean wind direction measured during the implementation of the initial flight plan, and to compare the mean wind direction to an assumed wind direction associated with the initial flight plan (e.g. perpendicular to the initial measurement area at the center thereof) by calculating the angle between the measured mean wind direction and the assumed wind direction.

If the angle is greater than a given coherence angle, e.g., less than 20°, preferably less than 10°, the trajectory correction module 58 is configured to establish an inconsistency of the measured wind direction with the positioning of the initial measurement area.

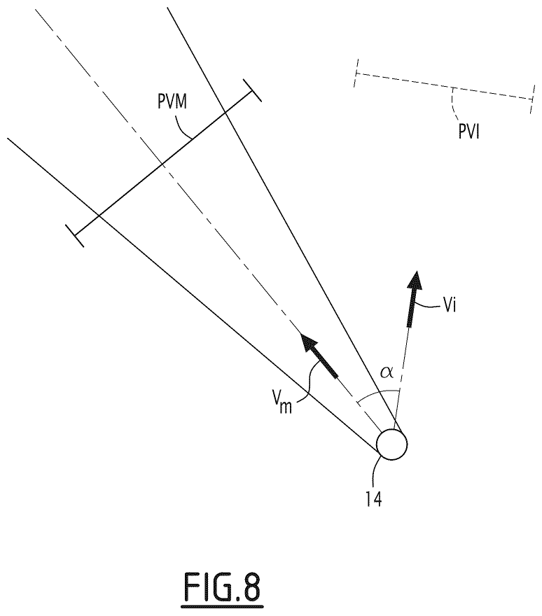

The trajectory correction module 58 is then configured to redefine a modified flight plan from the initial flight plan by changing the orientation of the measurement area to make said flight plan perpendicular at the center thereof to the measured mean wind direction, but preferably without changing the distance of the center of the measurement area from the source 14, as will be described below with reference to FIG. 8.

Conversely, if the angle difference is less than the given coherence angle, then the correction module 58 is configured to establish a coherence of the measured wind direction with the positioning of the initial measurement area.

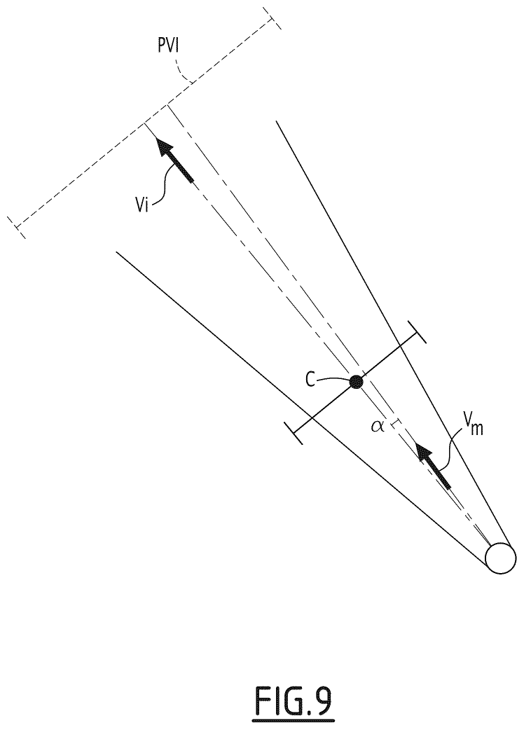

The trajectory correction module 58 is then configured to redefine a modified flight plan from the initial flight plan by moving the center of the measurement area closer to the source, without changing the orientation of the measurement area, as will be described below with reference to FIG. 9.

Referring to FIG. 1, the calculation system 24 here is located on the ground. The calculation system includes at least one computer 60 and a human-machine interface comprising a control organ 62 such as a keyboard, a mouse, and/or a touch screen, the human-machine interface also including a display 64, in particular a screen.

The computer 60 comprises, in a known manner, at least one processor 66 and a memory 68 including software modules capable of being executed by the processor 66 to perform functions. Alternatively, the computer 60 includes programmable logic components or dedicated integrated circuits, intended to perform the functions of the modules described below.

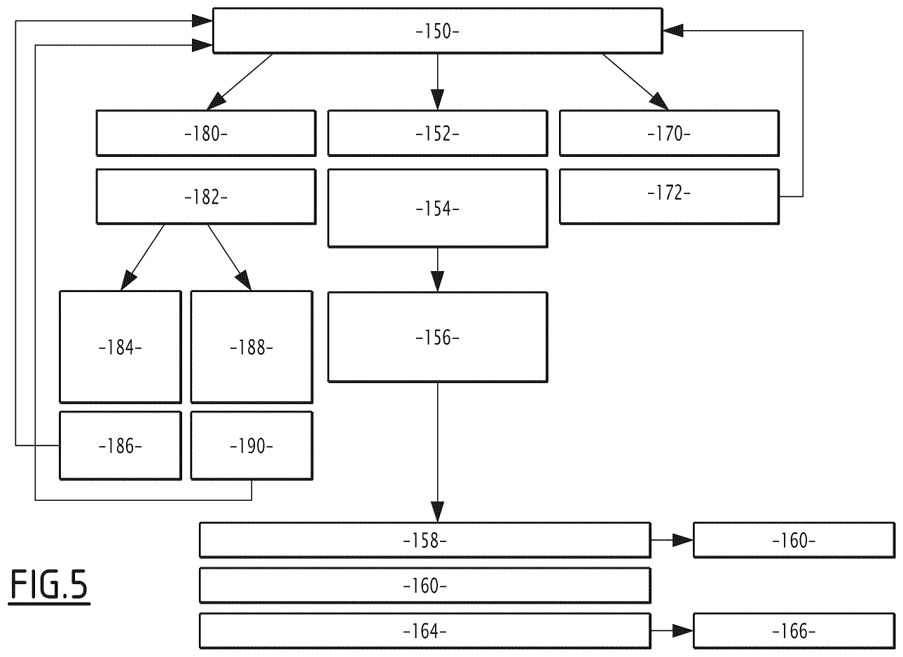

The calculation system 24 is, e.g., following the example described in patent application WO2021204941. In this example, referring to FIG. 1, the memory 68 contains a module 70 for obtaining and initially processing data representative of content of at least one gas, to calculate, on each parallel line 50, successive contents of at least one gas along the parallel line 50, an example of which is visible in FIG. 5.

The memory 68 further contains a module 72 for integrating the contents over each line 50 following the first direction D1, to obtain an integrated global content TGI on each line 50.

The memory 68 further contains a module 74 for interpolating a curve 75 of integrated global contents TGI along the second transverse direction D2 to the first direction D1 (see FIG. 6), from the integrated global contents TGI calculated over each line 50 and a module 76 for integrating the product of the integrated global content TGI according to the first direction by a wind speed V, the integration being performed following the second direction D2, to obtain a raw gas flow Qb circulating in the plume 16.

The memory 68 also contains a module 78 for determining a gas flow Qg emitted by the source 14 by correcting the raw gas flow Qb based on the conformation of the plume 16.

A first example of implementing the measurement method according to the invention, using the trajectory correction module 57 during the execution of the initial flight plan, will now be described. This method could also be implemented manually by an operator having access to the data representative of the content measured at each measurement point.

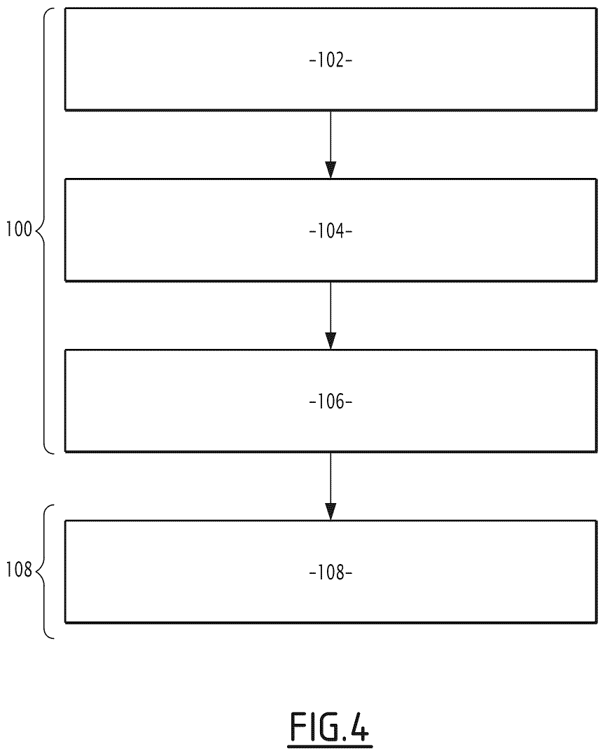

The method advantageously includes an initialization and wind detection phase 100, an example of which is illustrated in FIG. 4.

The phase 100 begins with a step 102 of acquiring wind data. During this step 102, the wind is measured to determine a direction and a low-frequency direction standard deviation, e.g. at a frequency greater than 20 seconds, in particular between 25 seconds and 45 seconds, to determine the wind direction, speed, and stability.

This step 102 can be performed directly with the drone 22 if it is equipped with a wind measurement sensor. In this case, the wind measurement is performed during an initial warm-up flight for a few minutes, or the wind measurement is performed directly before the implementation of the initial flight plan, during an ascending phase of the drone 22 at takeoff. Advantageously, the measurements are acquired by the acquisition and/or calculation module 56.

Alternatively, the wind direction is measured directly from the ground, e.g. using a wind measurement sensor without performing a flight of the drone 22.

Phase 100 then comprises a step 104 of calculating wind dispersion. The dispersion of wind measurements is calculated and adjusted, e.g. to a Gaussian distribution model to determine an initial wind direction value and an initial wind dispersion value.

Next, a step 106 of defining the flight plan is performed, advantageously by the acquisition and/or calculation module 56 using the wind direction, position, and possibly wind speed to determine the position of the center of the cross-section of the plume 16, over a line starting from the source 14 following the wind direction, at a distance that depends on the wind speed.

In addition, assumed width and height parameters of the cross-section of the plume 16 are automatically determined by calculation using dispersion models, and a vertical spacing between the measurement segments is then defined based on the assumed height of the cross-section of the plume 16.

The method then includes a phase 108 of implementing the initial flight plan.

The drone 22 is piloted by the control unit 42 to ascend to the altitude corresponding to that of the first measurement segment of the initial flight plan.

The drone 22 then traverses the measurement segment, measuring at each instant the data representative of the content in concentration.

If at least one value of the data representative of content is above the threshold value, the measurement segment is not the lowest segment of the plume. Subsequently, the trajectory correction module 57 then pilots the drone 22 to descend to a new measurement segment at a lower altitude. A sweep of the new measurement segment is performed, and these operations are repeated until a lower segment 50D is obtained with values of the data representative of content all below the background level threshold value.

The control unit 42 then ascends the drone 22 on a connection segment 52 to perform measurements over a first measurement segment with at least one value of the data representative of content above the content threshold value.

As specified hereinabove, on each measurement segment 50A of the initial flight plan, at each measurement of a data representative of content, as long as the value of the data representative of content is above the background level threshold value, then the trajectory correction module 57 moves the drone 22 to perform a new measurement of data representative of content over the same measurement segment 50A at a position adjacent to the position just measured.

This is particularly the case if the representative data at an end 51 of the initial measurement segment 50A provided in the initial flight plan have a value above the background level threshold value.

Referring to FIG. 3, the trajectory correction module 57 extends the initial measurement segment 51 over a section 51A beyond the end 51 of the initial measurement segment 50A, as long as the value of the data representative of content is above the background level threshold value.

When at least one measured datum representative of content is below the background level threshold value, preferably, when a plurality of successive data representative of content are below the background level threshold value, then the trajectory correction module 57 stops the movement of the drone 22 over the section 51A and stops the measurement by the measurement unit 34 over this measurement segment 50A. This stopping thereby defines a new lateral end 51B of the measurement segment 50A modified by the addition of the section 51A.

The trajectory correction module 57 then moves the drone 22 vertically following a connection segment 52 to reach a different altitude and to traverse a new measurement segment 50B adjacent to the previous measurement segment 50A.

The trajectory correction module 57 repeats the addition of new measurement segments 50B, even beyond the initially planned height of the highest measurement segment planned in the initial flight plan, as long as at least one value of data representative of content over the measurement segment 50B is above the background level threshold value.

When all the values of the data representative of content are below the background level threshold value over a given measurement segment, the trajectory correction module 57 stops the movement of the drone 22 and no longer performs measurements over new measurement segments 50B.

The entire section of the plume is then considered to have been measured.

The calculation system 24 is then implemented to calculate the concentrations at each measurement performed, and subsequently, the gas flow emitted by the source.

A first example of implementing the method, using the trajectory correction module 58 after the execution of the initial flight plan, will now be described.

Initially, the phase 100 described previously in FIG. 4 is implemented to determine the wind data and calculate the initial flight plan.

Then, referring to FIG. 5, a phase 150 of flying the drone 22 piloted by the control unit 42 is implemented to follow the initial flight plan. In this example, the modification module 57 is not necessarily activated during the execution of the initial flight plan.

At the end of the execution of the initial flight plan, the correction module 58 is then activated.

As mentioned above, in case 152 where the implementation of the initial flight plan following the initial measurement area has allowed values to be obtained of data representative of content above the background level threshold value, and no data representative of content above a saturation threshold value, at step 154, the trajectory correction module 58 calculates the position of a barycenter of the obtained values for the data representative of content.

The trajectory correction module 58 advantageously determines the wind direction at each measurement point, and possibly determines the measurement segments that are incomplete if the trajectory correction module 57 was not activated during the implementation of the initial flight plan.

As illustrated in FIG. 6, based on the calculated position B of the barycenter, advantageously the measured wind direction data V and the position of the incomplete segments, at step 156, the trajectory correction module 58 defines a modified flight plan PVM wherein the assumed center position of the section of the plume 16 (by placing it, e.g., at the determined barycenter position and preferably keeping the same distance to the source 54), the height, the width, the angular orientation of the measurement area and/or the vertical spacing of the measurement segments are modified compared to the initial flight plan PVI.

Then, during the same flight of the drone 22, without the drone 22 having landed, at step 158, the control unit 142 pilots the drone 22 again to follow the modified flight plan and obtain a first set 160 of measurements of data representative of the content over the modified measurement area.

Possibly, a new phase of flight plan modification is performed, repeating steps 152 to 156, to determine a new modified flight plan. At step 164, the control unit 142 pilots the drone 22 again, without it having landed, to follow the new modified flight plan and to obtain a new set 166 of measurements of data representative of content over the modified measurement area.

In a second case 170, referring to FIGS. 5 and 7, if data representative of content measured during the execution of the initial flight plan PVI has values above the saturation threshold value, the trajectory correction module 58 redefines at step 172, a modified flight plan PVM, over a modified measurement area further from the source 14 than the initial measurement area.

In a third case 180, when the values of the data representative of content are all below the background level threshold value, the trajectory correction module 58 determines, at step 182, a wind direction measured throughout the implementation of the initial flight plan.

The trajectory correction module 58 then determines a mean wind direction measured during the implementation of the initial flight plan, and compares said wind direction to an assumed wind direction for the definition of the initial flight plan, by calculating the angle a between the measured mean wind direction Vm and the assumed wind direction Vi.

Referring to FIG. 8, if the angle a is greater than a given coherence angle, e.g., less than 20°, preferably less than 10°, the trajectory correction module 58 determines at step 184, an inconsistency of the measured wind direction with the positioning of the initial measurement area of the initial flight plan PVI.

The trajectory correction module 58 redefines, at step 186, a flight plan modified from the initial flight plan by changing the orientation of the measurement area to make said orientation perpendicular at the center thereof to the measured mean wind direction Vm, but advantageously without changing the distance of the center of the measurement area of the modified flight plan PVM from the source 14.

Conversely, referring to FIG. 9, if the angle α is less than the given coherence angle, then the correction module 58 determines at step 188, a coherence of the measured wind direction Vm with the positioning of the initial measurement area of the initial flight plan PVI.

The trajectory correction module 58 redefines a modified flight plan PVM from the initial flight plan by moving the center C of the measurement area of the modified flight plan PVM closer to the source 14 compared to the initial flight plan PVI, without changing the orientation of the measurement area of the modified flight plan PVM relative to the source 14.

By means of the invention just described, it is possible to significantly increase the accuracy of the gas flow measurements performed, by correcting the flight plan initially defined for the drone 22 based on an assumption of the position of the plume 16.

This can be done during the execution of the initial flight plan by adding measurement points that might be missing in the sweep of the cross-section of the plume 16, either at the ends of each measurement segment or by adding additional measurement segments upwards and downwards.

The invention can also correct the flight plan after the execution of an initial flight plan, during the same flight of the drone 22, by redefining the position, dimensions, and orientation of the measurement area to fit exactly to the cross-section of the plume 16, being perpendicular and centered relative to this cross-section, and including it in its entirety.

This ensures that gas contents are measured throughout the entire cross-section of the plume 16. The accuracy of the calculated flow for the plume 16 is therefore much higher, and the measurement of the flow rate of emissions from the source 14 is more accurate.

The method according to the invention can be implemented very simply, either by means of an autonomous control unit 42 or by remotely piloting the control unit 42 to execute the steps of the method according to the invention.

In a variant, the execution of the initial flight plan by the drone 22 during the implementation of the method according to the invention can be interrupted by reaching a lower charge threshold on the batteries of the propulsion set 32. In this case, the drone 22 can be programmed to fly to a charging point or battery replacement point, then to resume the initial flight plan, or possibly a modified flight plan following the method according to the invention.

Advantageously, the drone 22 is programmable to automatically implement the method according to the invention at predefined successive times. These predefined times are, e.g. periodic, in particular daily, for monitoring a facility.

1. measuring method to measure contents of at least one gas emitted by a source in a plume propagating from the source through an atmosphere,
the method comprising:
collecting representative data, the representative data being representative of content of at least one gas in the atmosphere, the representative data being collected away from the source by flying a drone equipped with a representative data measurement unit;
defining an initial flight plan for the drone, the initial fight plan extending along an initial measurement area comprising a plurality of initial measurement segments;
implementing the initial fight plan by flying the drone along the initial flight plan and measuring representative data over at least one of the initial measurement segments;
during the implementing the initial flight plan and/or after the implementing the initial flight plan, generating a modified flight plan from the representative data measured over the at least one initial measurement segment, the modified flight plan including a plurality of modified measurement segments defining a modified measurement area; and
measuring representative data along the plurality of modified measurement segments of the modified measurement area.

2. The method according to claim 1, wherein the modified flight plan is generated during the implementing the initial flight plan, the drone subsequently flying along at least one modified measurement segment of the plurality of modified measurement segments, the at least one modified measurement segment replacing an existing initial measurement segment of the initial flight plan or adding to the existing initial measurement segment of the initial flight plan.

3. The method according to claim 2, wherein the at least one modified measurement segment extends the initial measurement segment beyond an end of the existing initial measurement segment until a value of at least one representative datum measured in the extension of the existing initial measurement segment is below a background level threshold value.

4. The method according to claim 2, further comprising a new measurement segment extending adjacent to an ongoing measurement segment, as long as at least one representative datum over the generated new measurement segment is above a background level threshold value.

5. The method according to claim 4, wherein the generated new measurement segment is located above or below the ongoing measurement segment.

6. The method according to claim 5, wherein a gap between the ongoing measurement segment and the generated new measurement segment is adjusted using an estimated distance from the source, and/or using an extent of segments previously flown by the drone.

7. The method according to claim 1, wherein the generating modified flight plan is implemented after completely executing the initial flight plan, the drone remaining in flight between an end of executing the initial flight plan and exectuing the modified flight plan.

8. The method according to claim 7, wherein, when the representative data measured over the initial measurement area have values within an exploitable value range, generating the modified flight plan includes determining a barycenter of the representative date values measured over the initial measurement area, then defining the measurement area from the position of the barycenter.

9. The method according to claim 7, wherein, when the representative data measured over the initial measurement area have at least one value above a saturation threshold value, generating the modified flight plan comprises positioning the modified measurement area further from the source than the initial measurement area.

10. The method according to claim 7, wherein, when the representative data measured over the initial measurement area all have a value below a background level threshold value, generating the modified flight plan includes estimating a wind direction measured from wind data measured during the flying the drone along the initial measurement area, and determining an angle between a measured wind direction and an initially assumed wind direction the initially assumed wind direction being assumed when defining the initial measurement area.

11. The method according to claim 10, wherein when the angle between the measured wind direction and the assumed wind direction is greater than a given coherence angle, generating the modified flight plan includes modifying an orientation of the modified measurement area with respect to the source compared to an orientation of the initial measurement area with respect to the source or wherein when the angle between the measured wind direction and the assumed wind direction is less than a given coherence angle, generating the modified flight plan includes defining a modified measurement area closer to the source than the initial measurement area.

12. The method according to claim 1, wherein the initial measurement segments and/or the modified measurement segments are straight lines, the initial measurement area and the modified measurement area being measurement planes.

13. The method according to claim 1, further comprising determining a position of the initial flight area from measured wind data and a dispersion calculation performed from the measured wind data.

14. The method according to claim 13, further comprising acquiring the measured wind data from a wind measurement sensor carried by the drone, during a warm-up phase of the drone and/or during an ascent of the drone to a starting altitude for implementing the at least one initial measurement segment of the initial flight plan.

15. The method according to claim 1, wherein defining the initial flight plan and generating the modified flight plan are implemented by a drone movement controller, the drone movement controller being on-board the drone or being at least partially remote from the drone.

16. The method according to claim 1, wherein representative data is collected at a frequency greater than 1 Hz.

17. An emission measuring method to measure emissions from a source, comprising implementing a measurement method according to claim 1, then calculating, using a flow calculator, of a gas flow emitted by the source from the representative data measured along the plurality of modified measurement segments of the modified measurement.

18. A drone configured to measure the contents of at least one gas emitted by a source in a plume propagating from the source through an atmosphere,
the drone including a representative data measurement unit configured to measure representative data, the representative data being representative of content of at least one gas in the atmosphere away from the source,
the drone comprising a drone movement controller configured to define an initial flight plan for the drone along an initial measurement area comprising a plurality of initial measurement segments;
the drone movement controller being configured to implement the initial fight plan by flying the drone along the initial flight plan to measure representative data over at least one initial measurement segment,
the drone movement controller being configured, during implementing the initial flight plan and/or after implementing the initial flight plan, to generate a modified flight plan from the representative data measured during the at least one initial measurement segment, the modified flight plan comprising a plurality of modified measurement segments defining a modified measurement area,
the drone movement controller being configured to control a flight of the drone to measure representative data over the plurality of modified measurement segments of the modified measurement area.

19. The method according to claim 8, comprising measuring wind data at each measurement point of a representative datum, then defining the measurement area from a mean wind direction calculated from the wind data determined at each measurement point of a representative datum.
